(19) United States
(12) Patent Application Publication
(10) Pub. No.: US 2026/0090706 A1
(43) Pub. Date: Apr. 2, 2026

(54) ENDOSCOPE HAVING AN INTEGRAL BENDING SECTION BODY WITH FLEXIBLY CONNECTED BENDING SEGMENTS

(71) Applicant: AMBU A/S, Ballerup (DK)

(72) Inventor: Frederik Clausager Vemb HANSEN, Copenhagen NV (DK)

(73) Assignee: AMBU A/S, Ballerup (DK)

(21) Appl. No.: 19/342,844

(22) Filed: Sep. 29, 2025

(30) Foreign Application Priority Data
Sep. 30, 2024 (EP) 24203450.2

(51) Int. Cl. A61B 1/005; A61B 1/00; A61B 1/012

(52) U.S. Cl. CPC A61B 1/0055; A61B 1/00045; A61B 1/00128; A61B 1/0057; A61B 1/012

(57) ABSTRACT

The present disclosure relates to an endoscope (2) including an interface (4); and an insertion cord (6) including an actively bendable bending section (10) which includes an integral bending section body (16, 16′) made of polymer material. The bending section body (16, 16′) includes segments (18, 18′) interconnected by hinges. The hinges (26, 26′) between adjacent segments (18, 18′) are formed by hinge protrusions (30, 30′) engaging in hinge recesses (32, 32′), and flexible connection portions (34, 34′) between the adjacent segments (18, 18′) provide an interconnection between the adjacent segments (18, 18′).

### CROSS-REFERENCE TO RELATED APPLICATIONS

This application claims priority from and the benefit of European Patent Application No. 24203450.2, filed Sep. 30, 2024; the disclosure of said application is incorporated by reference herein in its entirety.

### TECHNICAL FIELD

The present disclosure relates to an endoscope with a one-piece bending section, in a particular example the bending section comprising hinges formed by hinge protrusions engaging in hinge recesses.

### BACKGROUND

Endoscopes, which include specialized instruments such as bronchoscopes, arthroscopes, colonoscopes, laparoscopes, gastroscopes, duodenoscopes and ureteroscopes, are used for visual examination and diagnosis of hollow organs and body cavities, as well as to assist in surgery, e.g. for a targeted tissue sampling. Endoscopes can be reusable or disposable (i.e. single-use) and usually comprise a handle, an insertion cord, the insertion cord including an insertion tube, a bending section, and a distal tip unit. The insertion cord is configured to be inserted into the hollow organs and body cavities of a patient. A rotating force applied by a user to a wheel or a lever provided at the handle creates a pulling force acting on steering wires in an axial direction to bend the bending section and thus reorient the distal tip unit.

Single-use endoscopes may comprise a bending section body molded in one single piece or two single pieces of a polymer material. The bending section body of such a single-use endoscope comprises segments interconnected by bendable polymer strips. Two longitudinal polymer strips may be arranged approximately diametrically opposed with respect to a center axis of the bending section body to connect adjacent segments. A bending plane is defined by, and traverses, the polymer strips.

Metal bending sections are usually used in reusable endoscopes. The metal bending section may comprise segments riveted together, where the riveted portions form hinges. The metal bending section may also be laser-cut to form a one-part metal bending section. Such metal bending sections are rather insusceptible to compression loads, which means that high compression loads can be applied on them. Processing and manufacturing costs for metal bending sections are, however, too high for their commercial use in single-use endoscopes.

### SUMMARY

The objectives of the present technology are to mitigate or eliminate the disadvantages of the related art. In particular, an endoscope is disclosed which is designed for single-use, i.e. which has low manufacturing and assembly costs, and which has a bending section that suitably carries compression loads.

In a first aspect, the present technology provides an endoscope comprising an actively bendable single-piece polymeric bending section body comprising segments interconnected by flexible connection portions and hinges provided between adjacent segments, the hinges comprising hinge protrusions configured to separably engage hinge recesses. Segments may also be referred to as bending segments, due to the segments being part of the bending section.

In one embodiment according to the first aspect, pairs of the segments, which are adjacent, may be interconnected by four flexible connection portions. Each hinge may comprise diametrically opposite hinge interfaces. Each hinge interface comprises a hinge protrusion and a hinge recess that is detached from, but under certain conditions abuts, the hinge protrusion.

The bending section body may be described as having a compression resistant hinge design. The bending section body is preferably manufactured such that the hinge protrusions are spaced or distanced from the hinge recesses. During assembly of the endoscope, steering wires are pretensioned, i.e. pulled, resulting in a compression load on the bending section body, said compression load leading to an elastic deformation, i.e. some kind of pre-bending of the flexible connection portions and leading to the hinge protrusions abutting the hinge recesses. Via the pretension provided to the steering wires, inherent elasticity is taken from the bending section body, leading to the hinge protrusions contacting or touching, and thus engaging the hinge recesses. Preferably, the hinge protrusions are pressed into the hinge recesses until a kind of hysteresis state is reached, which removes slack. This makes it possible that when an operator operates the endoscope, in particular pulls the steering wires, the bending section body is directly bent in the desired direction, i.e. the bending section body does not have to be elastically deformed or compressed first to remove the slack. It is thus preferred that in an assembled state of the endoscope the hinge protrusion and the hinge recess forming a hinge interface abut and thus engage each other.

The endoscope may comprise a positioning interface and an insertion cord configured to be inserted into a patient's body cavity and comprising the actively bendable bending section. The insertion cord is connected to a distal end of the positioning interface, which may be referred to as a handle of the endoscope. The handle may comprise one or more control actuators, including manual control actuators. Alternatively, the positioning interface may be detachably connected to a robotic arm. The positioning interface is configured to control a position of the insertion cord. Preferably, the insertion cord comprises an insertion tube and a distal tip unit, the actively bendable bending section extending between, and connecting, the insertion tube and the distal tip unit.

The bending section comprises an integral bending section body made of polymer material. The bending section body is thus an integral part being formed as a single piece from, preferably thermoplastic, polymer material. The bending section body is preferably manufactured in an injection-molding process. It is to be understood that the bending section comprises preferably only one such bending section body. However, the present disclosure is not limited to this embodiment, and in an alternative, a plurality of integral bending section bodies connected to each other may be provided.

The bending section body comprises the plurality of interconnected bending segments. I.e. the plurality of bending segments are integrally connected with each other, thereby forming in combination the integral bending section body, and are thus not separate or independent parts. In particular, the plurality of interconnected bending segments may comprise a proximal end segment, a plurality of intermediate segments and a distal end segment. The proximal end segment of the bending section body is the proximal-most bending segment of the plurality of interconnected bending segments and is preferably adapted to be connected to the insertion tube of the insertion cord. The distal end segment of the bending section body is the distal-most bending segment of the plurality of interconnected bending segments and is preferably adapted to be connected to the distal tip unit of the insertion cord. The proximal end segment and the distal end segment are preferably designed or adapted such that they may be suitably connected to a remainder of the insertion cord and thus provide suitable interface parts fitting to the remainder of the insertion cord.

The hinges between adjacent bending segments are formed by hinge protrusions engaging in hinge recesses. The hinge protrusions and hinge recesses may be formed so as to allow a rolling movement or motion in an interface between the hinge protrusions and the hinge recesses, resulting in a desired bending movement of two adjacent bending segments. In other words, two adjacent bending segments are preferably not connected via hinges or hinge members but have an unconnected hinge interface/unconnected hinge interfaces.

Hinges between two adjacent bending segments may include a hinge between the proximal end segment and an adjacent intermediate segment, hinges between adjacent intermediate segments, and a hinge between the distal end segment and an adjacent intermediate segment. Between adjacent bending segments of the bending section body usually two hinge interfaces are provided, i.e. two hinge protrusions engaging in two hinge recesses. The hinge interfaces are preferably arranged approximately diametrically opposed with respect to a center axis of the bending section body. The intermediate segments may be identically formed, i.e. each intermediate segment may comprise a hinge protrusion or hinge protrusions on one axial end and may comprise a hinge recess or hinge recesses on the other axial end. Alternatively, the bending section body may have different intermediate segments arranged one after the other in an axial direction of the bending section body. For example, a first intermediate segment may only have hinge protrusions and a second intermediate segment may only have hinge recesses for engagement with the hinge protrusions of the first intermediate segment.

The hinge protrusion may be designated as convex compression limiting part or portion and the hinge recess may be designated as concave compression limiting part or portion. The compression resistant hinge design according to the present disclosure preferably prevents any collapse of hinges during assembly and thus prevents any S-shaped deformation of the bending section body, coming along with preferably no impairment of the bending performance in an endoscopic procedure performed by an operator. The compression limiting parts or portions preferably abut each other after a pre-tensioning of steering wires was performed during assembly of the endoscope. This means that after assembly and during use of the endoscope, there is preferably always contact between the two compression limiting parts. Thereby, it is avoided that the compression limiting parts or portions need to be moved axially to compress and decompress the bending section body during each bending cycle.

The present disclosure makes it possible that the hinge protrusion, which may be designated as the convex compression limiting part or portion, may be structurally optimized to withstand compression forces. E.g., the hinge protrusion may be a short and wide protrusion suitable to carry compression forces. On the other hand the flexible connection portions may be structurally optimized to provide a desired, especially high, flexibility. E.g., the flexible connection portions may be long and thin material bridges between two adjacent bending segments. According to the present disclosure, it is thus possible to optimize the two functions “Compression Resistance” and “Flexibility” independently since the two functions are split into two separate features. In particular, the “Compression Resistance” can be optimized by suitably designing the hinge interface, in particular the hinge protrusion and the hinge recess, and the “Flexibility” can be optimized by suitably designing the flexible connection portions.

A major advantage of the bending section body according to the present disclosure is that both functions “Compression Resistance” and “Flexibility” can be optimized independently without having to increase the thickness, i.e. the extension in radial direction, of the bending section body. E.g., the flexibility of the flexible connection portions may be suitably adjusted by selecting a suitable length in axial direction and a suitable width in circumferential direction, and the compression resistance may be suitably adjusted by selecting a suitable, rather short, length in axial direction and a suitable, rather big, width in the circumferential direction. Therefore, a ratio between inner diameter ID and outer diameter OD of the bending section body can be optimized. Note that it is desirable in endoscopic procedures to have an outer diameter OD of the bending section body which is as small or thin as possible, in order to be able to insert the insertion cord in a respective body cavity, and to have an inner diameter ID which is as big as possible, in order to be able to provide a working channel tube inside the bending section body having an inner diameter which is as big or large as possible.

In traditional single-use endoscopes, with respect to the foil hinges, which are short strips of bendable bridges of material between adjacent bending segments, usually a compromise needs to be found as follows: On the one hand, the hinges should be thick enough to withstand compression forces and thus to avoid the S-shaped deformation, which may also be referred to as “snaking”, of the bending section. On the other hand, the hinges should be as thin as possible to reduce stress in the polymer material and to ensure low forces during bending.

Preferably, the hinge between two adjacent bending segments comprises two diametrically opposed hinge interfaces, each hinge interface being formed by a hinge protrusion engaging in a hinge recess. In other words, preferably two hinge protrusions engage in two hinge recesses, in particular a first hinge protrusion engages in a first hinge recess (first hinge interface) and a second hinge protrusion engages in a second hinge recess (second hinge interface), between two adjacent bending segments.

Each hinge interface is encircled or surrounded by two flexible connection portions. In other words, there may be provided a first flexible connection portion clockwise in circumferential direction next to the hinge interface and a second flexible connection portion anticlockwise in circumferential direction next to the hinge interface. Preferably, the flexible connection portions encircling or surrounding the hinge interface are arranged directly next to or adjacent to the hinge interface. An arrangement directly next to or adjacent to the hinge interface means that the flexible connection portions are spaced by less than 90° in circumferential direction, preferably by less than 45° in circumferential direction, from the hinge interface, in particular from a center of the hinge interface. Each flexible connection portion is preferably connected to both adjacent bending segments. In case of the provision of two diametrically opposed hinge interfaces, there are thus preferably provided four flexible connection portions, wherein two flexible connection portions surround or encircle the first hinge interface and two flexible connection portions surround or encircle the second hinge interface.

The flexible connection portions may be spaced between 20° and 60° in circumferential direction, meaning that one of the flexible connection portions is between 20° and 60° from the center of the hinge interface in a clockwise direction, and the other flexible connection portion may be spaced between 20° and 60° from the center of the hinge interface in an anticlockwise direction.

The flexible connection portions may be curved or angled portions having a bend point or inflexion point. The bend point or inflexion point may be provided halfway in between the two adjacent bending segments. In particular, it may be provided that the flexible connection portions extend from one bending segment of the two adjacent bending segments, both in axial direction and in circumferential direction away from the hinge interface, towards the bend point or inflexion point, from which the flexible connection portions extend, both in axial direction and in circumferential direction towards the hinge interface, to the other bending segment of the two adjacent bending segments.

Two flexible connection portions surrounding or encircling a hinge interface may be symmetrically identical with respect to a bending plane defined by the hinge interface, in particular by the two diametrically opposed hinge interfaces.

The flexible connection portions may elastically deform when the steering wires are pretensioned, i.e. pulled during assembly of the endoscope. In particular, the flexible connection portions are configured to deform elastically when the hinge protrusions approach and abut the hinge recesses during pretensioning of the steering wires. E.g., a portion of the flexible connection portion upstream of the bend point or inflexion point may approach a portion of the flexible connection portion downstream of the bend point or inflexion point, the portions thus axially approaching each other while being bent around the bend point or inflexion point.

Two flexible connection portions surrounding or encircling a hinge interface has the following advantages: in the assembled state of the endoscope, when the operator performs a bending movement of the bending section, one of the two flexible connection portions will be further bent, i.e. compressed, while the other flexible connection portion will be stretched. The two flexible portions surrounding or encircling the hinge interface advantageously exert a force on the two adjacent bending segments, which will bring the bending section back to a neutral non-bent position when there is no pull on one of the steering wires in addition to the factory pre-tensioning. This may be referred to as “self-righting.” The flexible connection portions may thus be configured to apply a force to the bending section body which brings the bending section body back to a neutral position, in particular to urge or force the bending section body back to the neutral position when there is no pull on one of the steering wires.

According to an alternative embodiment, the flexible connection portions do not surround or encircle the hinge interface formed by the hinge protrusion engaging the hinge recess but are spaced from the hinge interface. E.g., two flexible connection portions may be provided. Each flexible connection portion may especially preferred be spaced by around 90° in the circumferential direction from the hinge interface(s). In the present embodiment, the flexible connection portions providing the interconnection between two adjacent bending segments do not serve to urge or force the bending section body back into a neutral, non-bent position when there is—compared to the factory pretensioning—no additional pull on one of the steering wires. In particular, the flexible connection portions may serve another function—to provide guiding apertures for steering wires in the assembled state of the endoscope, i.e. in a state in which the hinge protrusions abut the hinge recesses.

According to the alternative embodiment, each flexible connection portion may comprise two apertures and a predetermined bend or kink point or line or portion arranged in the axial direction between the two guiding apertures. There may be additional kink points or lines on both sides of the apertures to control the behavior under compression. In other words, in the axial direction, a first aperture of the two apertures may be closer to one bending segment of the two adjacent bending segments, and a second aperture of the two apertures may be closer to the other bending segment of the two adjacent bending segments, with the predetermined bend or kink point or line or portion arranged axially in between the two apertures. In still other words, each flexible connection portion may be described as comprising two longitudinal or axial webs essentially arranged in parallel with respect to each other, with a cross web forming the predetermined bend or kink point or line or portion and connecting the two longitudinal or axial webs, such that the two apertures are formed.

The bending section body may comprise two steering wire lumens, namely a first steering wire lumen and a second steering wire lumen, each steering wire lumen being preferably a rather small lumen and configured and provided to accommodate, respectively, a first steering wire and a second steering wire. The steering wire lumen may be provided in addition to the one or more above-mentioned inner lumen. The steering wire lumen may be spaced by around 90° in circumferential direction from the diametrically opposed hinge interfaces. A steering wire lumen may be closed or open. Open steering wire lumens are connected to the inner lumen.

According to the alternative embodiment, when the steering wires are pretensioned, i.e. pulled during assembly of the endoscope, the hinge protrusion(s) abut the hinge recesse(s) and the flexible connection portions bend or kink radially inwards at the predetermined bend or kink point or line or portion, wherein via the inward bending or kinking of the flexible connection portions, the guiding apertures for the steering wires are formed. In a variation, the flexible connection portions could bend outward instead of inwards. This would have the effect that the working channel is not being squeezed inside the bending section during bending. In this variation the flexible connection may preferably be designed to have rounded shapes in order to not affect the tissue when moved in body cavities. The bending cover provided on the external side of the bending section would also limit any interference with the tissue.

Preferably, the apertures are oval, i.e. have an oval shape, thereby enabling the steering wires to move freely, in particular independently of an actual bending angle of the bending section. In particular, it has turned out that in order to obtain round guiding apertures in a top view onto the bending section body, the apertures provided in the flexible connection portions are preferably oval. I.e. by bending or kinking the flexible connection portions radially inwards at the predetermined bend or kink point or line or portion, the oval apertures form essentially round or circular guiding apertures, seen in the top view. The apertures could also be elongated slots.

In the alternative embodiment, the steering wire lumens may be omitted due to the provision of the guiding apertures for the steering wires. Therefore, according to the alternative embodiment, preferably no additional steering wire lumen are provided. Thereby, the need for long thin molding cores, in the molding process of the bending section body may be avoided. This may reduce the manufacturing costs. This may also enable a larger ratio between the inner diameter and the outer diameter of the bending section body.

The first and the alternative embodiments may comprise the same positioning interface, described above. The bending section body in both embodiments may comprise one large inner lumen for accommodating a working channel tube, potentially also for further tubes or electrical cables. Alternatively, there may be one large inner lumen, especially for the working channel tube, and another inner lumen, e.g. for electrical cables.

The bending section body in the first and the alternative embodiments may comprise the same polymer material, which is, preferably, a rather hard and stiff polymer material, in particular a less flexible polymer material. In particular, the polymer material of the bending section body may be optimized to provide a suitable torsional stiffness of the bending section body. The suggested designs may reduce the torsional stiffness of the bending section body relative to traditional foil hinge designs. However, torsional stiffness may be regained by manufacturing, in particular molding, the bending section body with a harder and stiffer material, e.g. polypropylene. The polymer may comprise, or consist substantially of, polypropylene.

The present disclosure also relates to a system comprising an endoscope as defined above and a monitor connected to the endoscope.

### BRIEF DESCRIPTION OF THE FIGURES

The above-mentioned embodiments and variations, features and advantages thereof will be further elucidated by the following illustrative and nonlimiting detailed description of embodiments disclosed herein with reference to the appended drawings, wherein:

FIG. 1 is a perspective view showing an endoscope according to the present disclosure;

FIG. 2 is a perspective view of a bending section of the endoscope;

FIG. 3 is a cross-sectional view through the bending section of FIG. 2;

FIG. 4 is a detailed side view of a portion of the bending section of FIG. 2;

FIG. 5A is a perspective view of a bending section body of the bending section of FIG. 2 in a non-assembled state of the endoscope;

FIG. 5B is a detailed side view of hinges and flexible connections in a non-assembled state of the bending section, similar to FIG. 4.

FIG. 6A is a perspective view of the bending section body of the bending section of FIG. 2 in an assembled state of the endoscope;

FIG. 6B is a detailed side view of hinges and flexible connections in an assembled state of the bending section.

FIG. 7 is a perspective view of a bending section body according to an alternative embodiment of the present disclosure;

FIG. 8 is a detailed perspective view of a flexible connection portion of the bending section body of FIG. 7;

FIG. 9A is a variation of the flexible connection design in the alternative embodiment, shown in a non-assembled state of the bending section.

FIG. 9B is a close up of the flexible connection in FIG. 9A.

FIG. 10 a cross-sectional view of the bending section of FIG. 9A but shown in an assembled state of the bending section.

In the drawings, corresponding reference characters indicate corresponding parts, functions, and features throughout the several views. The drawings are not necessarily to scale, and certain features may be exaggerated in order to better illustrate and explain the disclosed embodiments.

### DETAILED DESCRIPTION

An endoscope according to the present technology is preferably a low-cost, lightweight, single-use endoscope, which is intended to be disposed after use in a single patient. This means that the endoscope is preferably optimized for one single use, therefore it has a limited number of elements, which are preferably manufactured with a low-cost material (polymer/plastic/resin) in preferably a low-cost manufacturing process (plastic/injection molding) and which can be easily assembled. Unlike reusable endoscopes, the endoscope according to the present technology is configured to be used with a single patient and then discarded, therefore the costly components necessary for reusable endoscopes to withstand rather aggressive cleaning or sterilization processes and general harsh handling over the life cycle of the endoscope are avoided.

An endoscope according to the present technology is preferably a small-diameter endoscope, e.g. 3.4 mm in diameter or less, in particular a bronchoscope, a ureteroscope or a cholangioscope (used as a baby endoscope in combination with a duodenoscope), etc. However, the present disclosure is neither limited to the endoscope being any one of the mentioned specific small-diameter endoscopes nor limited to the endoscope being a small-diameter endoscope. The endoscope according to the present technology may, advantageously, be another specialized medical instrument like a duodenoscope, bronchoscope, arthroscope, colonoscope, laparoscope, gastroscope, etc.

An endoscope according to the present technology is preferably a one-plane bending endoscope configured to bend in two opposite directions (e.g. up-down). Alternatively, the endoscope may be a two-plane bending endoscope configured to bend in four directions (e.g. up-down and left-right). The endoscope 2 is preferably a single-use endoscope and is preferably, but not necessarily, a small-diameter endoscope, such as an ureteroscope.

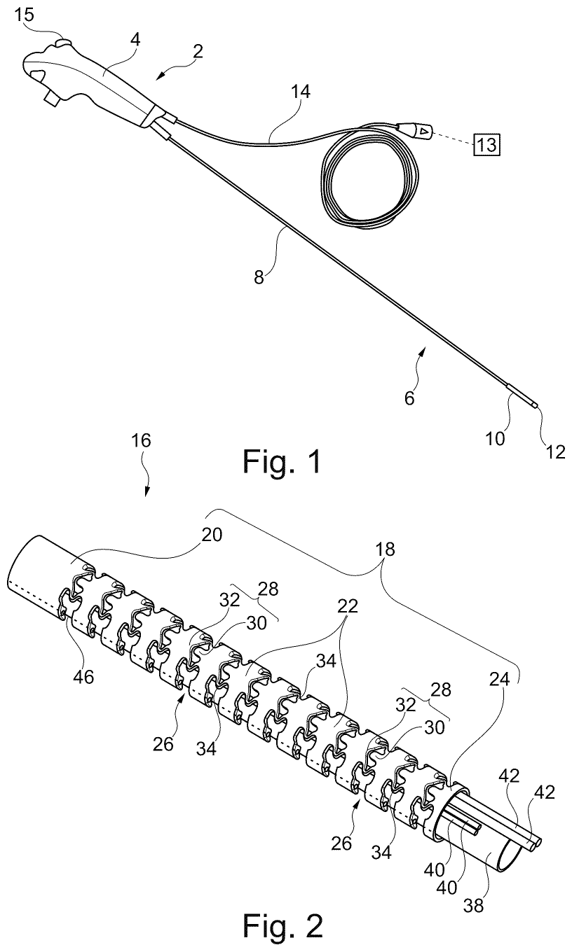

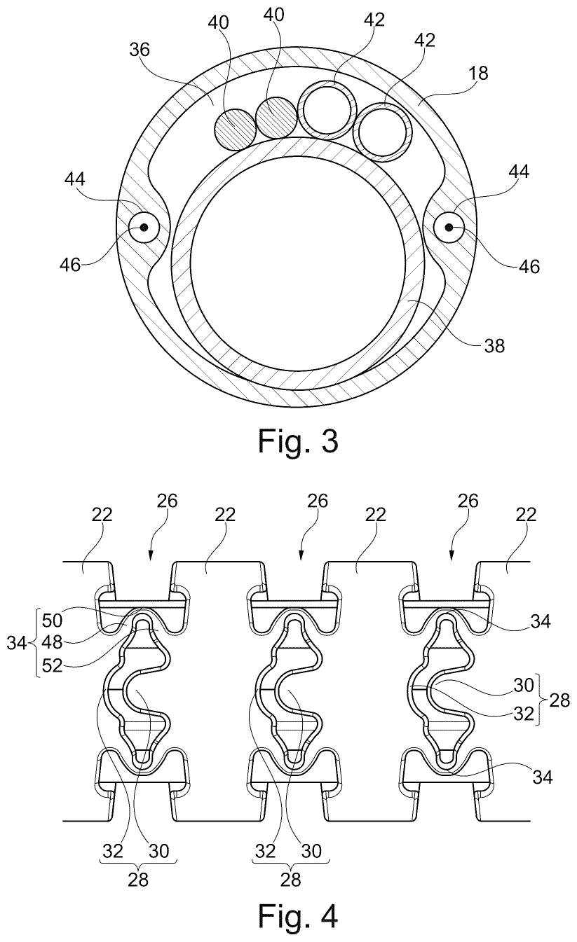

An endoscope 2 according to the present technology is shown in FIG. 1. The endoscope 2 comprises a positioning interface, exemplified as a handle 4, and an insertion cord 6 connected to and extending from the handle 4. A cable 14 is configured to connect the endoscope 2 to a monitor 13. The handle 4 is designed to be held by a user/physician and configured to accommodate operating parts of the endoscope 2. An operating unit, or steering actuator, 15, is supported by the handle and connected to steering wires 46 (shown in FIGS. 2 and 3). The steering actuator 15 may comprise, as shown, a lever extending through a housing of the handle 4 and comprising a user surface configured to receive a user input configured to rotate the steering actuator about a rotation axis. The steering actuator 15 may also comprise a wheel, instead of a lever, supported by the handle 4 and configured to rotate about a rotation axis. The steering actuator may comprise an internal roller connected to the wheel or the lever and configured to rotate responsive the user input.

The insertion cord 6, which is configured to be inserted into a patient's body cavity, comprises, extending distally in order, an insertion tube 8, an actively bendable bending section 10 and a distal tip unit 12. The bending section 10 may comprise a flexible sleeve or cover over a bending section body 16 (see FIG. 2). The steering wires 46, connected to the steering actuator 15, may extend through the insertion tube 8 and the bending section 10. Although FIG. 1 shows a one-plane bending endoscope, the endoscope 2 may also be a two-plane bending endoscope, and thus be configured for bending in a first bending plane and in a second bending plane. The second bending plane is preferably perpendicular to the first bending plane. The bending section body 16 of the two-plane bending endoscope may comprises hinges in the second bending plane that are the same or different from the hinges for the first bending plane. In one example, the second bending plane hinges are polymer strips that function as hinges and connecting portions. In another example, the second bending plane hinges have the same structure, and may be identical to, the hinges and connecting portions of the first bending plane hinges and connecting portions described with reference to FIGS. 2-10.

At the distal tip unit 12, image capturing means, such as an image sensor, e.g. a miniature video camera, and illuminating means, such as light-emitting diodes or optical fibers, are arranged/installed, such that the patient's body cavity can be illuminated and inspected. An image captured by the image capturing means can be shown on the monitor 13. The optical fibers may be connected to a proximal source of light located in the distal tip unit, at the positioning interface, or physically separately from the endoscope. The distal tip unit 12 may be tilted/reoriented by rotating the steering actuator 15 to pull the steering wires 46 and thus control the bending movement of the bending section 10 to tilt the distal tip unit 12.

FIG. 2 shows a perspective view of the bending section body 16, which is an integral, single piece of polymer material, preferably manufactured by injection molding a thermoplastic polymer material, especially preferred a rather hard and stiff polymer material like polypropylene or polyoxymethylene (POM). The bending section body 16 comprises a plurality of interconnected bending segments 18 including a proximal end segment 20, a plurality of intermediate segments 22 and a distal end segment 24. The proximal end segment 20 is connected to the insertion tube 8. The distal end segment 24 is connected to the distal tip unit 12. A hinge 26 between adjacent bending segments 18 is formed by two diametrically opposed hinge interfaces 28, wherein each hinge interface 28 comprises a hinge protrusion 30 and a hinge recess 32. Flexible connection portions 34 provide an interconnection between adjacent bending segments 18.

With reference also to FIG. 3, which is a cross-sectional view through the bending section 10, in particular through a bending segment 18 of the bending section 10, in addition to FIG. 2, the bending section body 16 comprises or forms one large inner lumen 36, in which a large working channel tube 38, electrical cables 40, and potentially further tubes 42 like a rinsing tube or an insufflation tube are accommodated. The bending section body 16 further comprises or forms two small steering wire lumens 44, in which the steering wires 46 are accommodated and guided through. The steering wire lumens 44 are arranged diametrically opposed with respect to each other and are spaced by around 90° in a circumferential direction from the hinge interfaces 28.

With reference also to FIG. 4, which is a detailed side view of a portion of the bending section 10 shown in FIG. 2, in addition to FIG. 2, a state during assembly of the endoscope 2 is shown, in which the working channel tube 38, the electrical cables 40 and the tubes 42 are already arranged in and guided through the large inner lumen 36, and in which the steering wires 46 are already arranged in and guided through the steering wire lumen 44. However, the steering wires 46 are not yet fixed to the distal end segment 24 and the steering wires 46 thus also have not yet been pretensioned, i.e. pulled.

In the state during assembly shown in FIGS. 2 and 4 the hinge protrusions 30 are spaced from the hinge recesses 32, i.e. the hinge protrusions 30 do not yet engage in the hinge recesses 32 and are thus not in contact with them, respectively do not abut them. Each hinge interface 28 formed by the hinge recess 32 and the associated hinge protrusion 30, which is provided to be engaged with the respective hinge recess 32 in the assembled state of the endoscope 2, is encircled or surrounded by two flexible connection portions 34. One flexible connection portion 34 is arranged directly next to, i.e. adjacent the hinge interface 28 clockwise in circumferential direction and one flexible connection portion 34 is arranged directly next to, i.e. adjacent the hinge interface 28 anticlockwise in circumferential direction. Both flexible connection portions 34 are spaced by less than 45° in circumferential direction from a center of the hinge interface 28. Each flexible connection portion 34 is connected to both adjacent bending segments 22. In particular, an upstream portion 48 of each flexible connection portion 34 extends from one bending segment 22 of two adjacent bending segments 22, both in axial direction and in circumferential direction away from the hinge interface 28, towards a bend point or inflexion point 50, from which a downstream portion 52 extends, both in axial direction and in circumferential direction towards the hinge interface 28, to the other bending segment 22 of the two adjacent bending segments 22. The flexible connection portions 34 are thus curved or angled and have the bend point or inflexion point 50 in axial direction halfway in between the two adjacent bending segments 22. The two flexible connection portions 34 surrounding or encircling the hinge interface 28 are symmetrically identical with respect to a bending plane defined by two diametrically opposed hinge interfaces 28 of the hinge 26. Since each hinge 26 comprises two diametrically opposed hinge interfaces 28, there are provided four flexible connection portions 34 for each hinge 26.

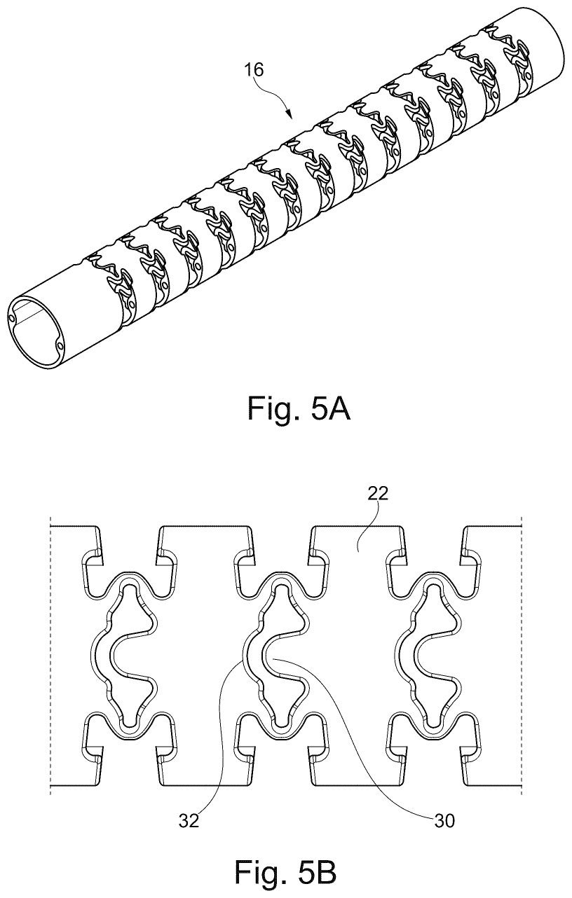

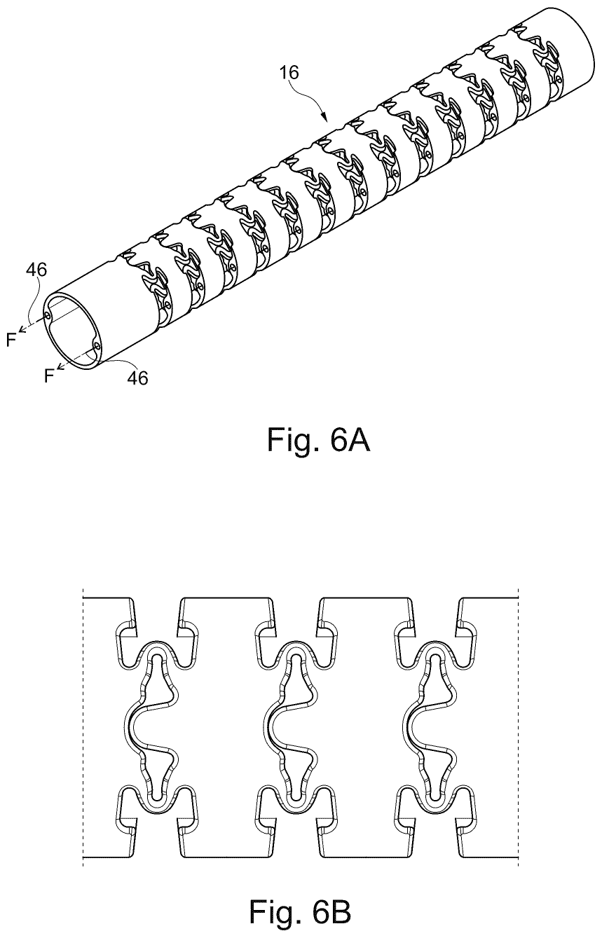

FIGS. 5A and 5B show a perspective view and a detailed side view of the bending section body 16 in the same state as in FIG. 2 and FIG. 4, i.e. in a state in which the hinge protrusions 30 are spaced from the hinge recesses. FIGS. 6A and 6B show a perspective view and a detailed side view of the bending section body 16 in an assembled state of the endoscope 2. For illustration purposes, all elements of the endoscope 2 except for the bending section body 16 and the steering wires 46 were removed in FIG. 6A. During assembly of the endoscope 2, the steering wires 46 are pretensioned, i.e. pulled, cf. force F in FIG. 6A, resulting in a compression load on the bending section body 16. The compression load leads to an elastic deformation of the flexible connection portions 34, in particular to the upstream portion 48 and the downstream portion 52 approaching each other and bending around the bend point or inflexion point 50, and leads also to the hinge protrusions 30 abutting or contacting or touching or engaging the hinge recesses 32. FIGS. 6A and 6B show the assembled state of the endoscope 2, in which the hinge protrusions 30 engage in the hinge recesses 32 and the flexible connection portions 34 have already been elastically deformed. When an operator pulls the steering wires 46 in the state shown in FIGS. 6A and 6B, the bending section 10 is directly bent in the desired direction (e.g. up or down), since the bending section body 16 does not have to be compressed first. In particular, the hinge protrusions 30 and the hinge recesses are formed so as to allow a rolling movement or motion in the hinge interfaces 28 between the hinge protrusions 30 and the hinge recesses 32. One of the flexible connection portions 34 surrounding the hinge interface 28 will be compressed, while the other one of the flexible connection portions 34 surrounding the hinge interface 28 will be stretched. When the operator stops pulling the steering wires 46, the flexible connection portions 34 bring the bending section body 16 back to a neutral, non-bent position due to their inherent material elasticity/flexibility.

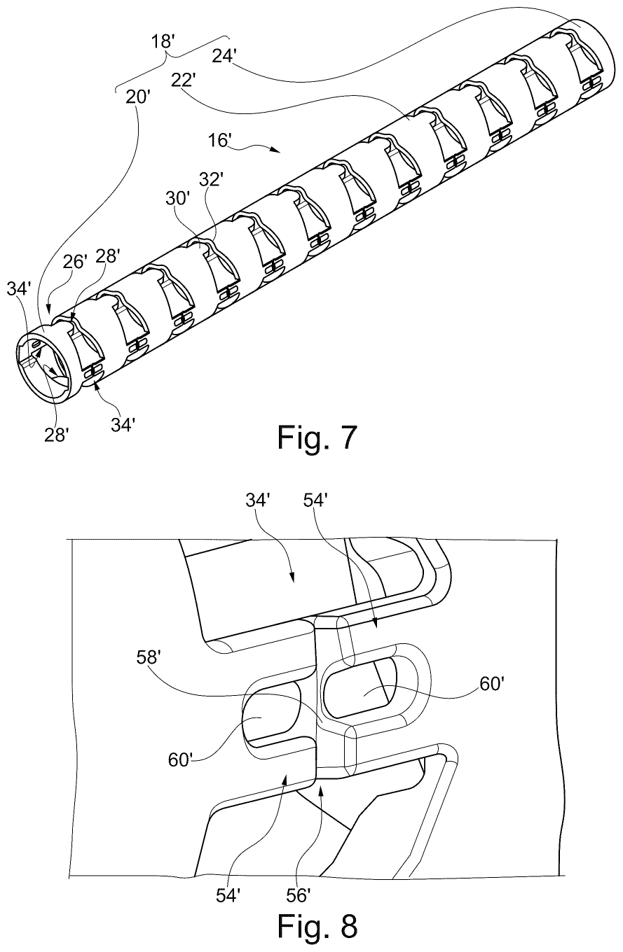

FIG. 7 shows a perspective view of a bending section body 16′ according to an alternative embodiment of the present technology. The bending section body 16′ is an integral piece of polymer material, preferably manufactured by injection molding a thermoplastic polymer material. The polymer may be a rather hard and stiff polymer material like polypropylene, or a more flexible polymer material like POM. The choice of polymer material may depend on the specific design of the flexible connection portions. The bending section body 16′ comprises a plurality of interconnected bending segments 18′ including a proximal end segment 20′, a plurality of intermediate segments 22′ and a distal end segment 24′. The proximal end segment 20′ is connected to the insertion tube 8. The distal end segment 24′ is connected to the distal tip unit 12. A hinge 26′ between adjacent bending segments 18′ is formed by two diametrically opposed hinge interfaces 28′, wherein each hinge interface 28′ comprises a hinge protrusion 30′ and a hinge recess 32′. Two flexible connection portions 34′ provide an interconnection between adjacent bending segments 18′.

The two flexible connection portions 34′ according to the alternative embodiment do not surround or encircle the hinge interface 28′ formed by the hinge protrusion 30′ engaging the hinge recess 32′, but are spaced from the hinge interface 28′. The two flexible connection portions 34′ are arranged diametrically opposed with respect to each other and are spaced by around 90° in the circumferential direction from the hinge interfaces 28′. With reference also to FIG. 8, which shows a detailed perspective view of one flexible connection portion 34′, each flexible connection portion 34′ comprises two longitudinal or axial webs 54′ arranged essentially in parallel with respect to each other, with a cross web 56′ connecting the two longitudinal or axial webs 54′ and essentially forming or providing a predetermined bend or kink line 58′. Two oval apertures 60′ are formed by the two longitudinal axial webs 54′ and the cross web 56′, wherein the two apertures 60′ are arranged next to, i.e. adjacent each other in the axial direction of the bending section body 16′.

When the steering wires 46 are pretensioned, i.e. pulled during assembly of the endoscope 2 the hinge protrusions 30′ engage, i.e. abut, the hinge recesses 32′ and the two flexible connection portions 34′ deform elastically. In particular, the two flexible connection portions 34′ bend or kink radially inwards at the predetermined bend or kink line 58′ of the cross web 56′. Via the inward bending or kinking of the flexible connection portions 34′ the two oval apertures 60′ form and provide guiding apertures for the steering wires 46.

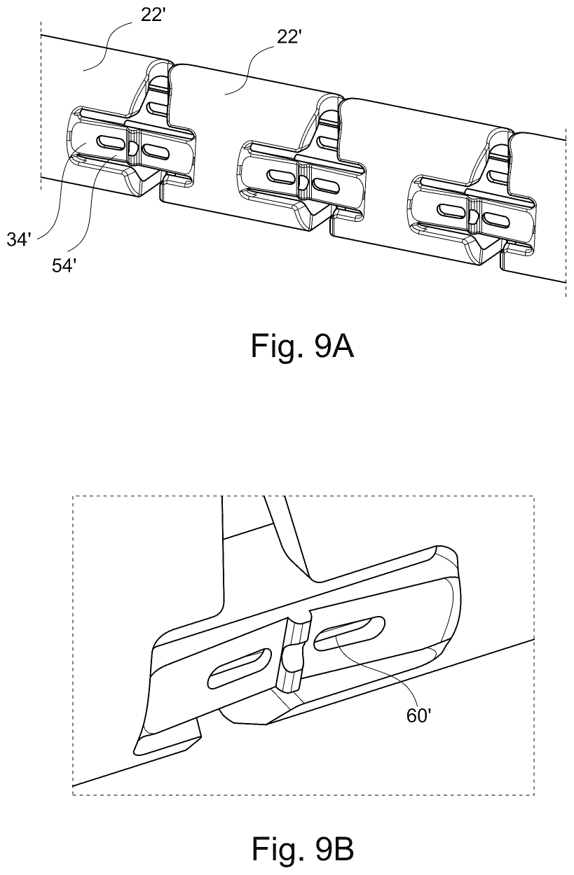

FIGS. 9A and 9B show a variation of the flexible connection design in the alternative embodiment. This is shown in a non-assembled state of the bending section. In this variation the flexible connection portion 34′ comprises one longitudinal or axial web 54′ connecting two neighboring segments 22′. This web 54′ is made as a thin film-like layer, which will bend when compressed. One advantage of this design is, that it may be simpler and faster to mold, when the bend or kink line shown in FIG. 8, is not included.

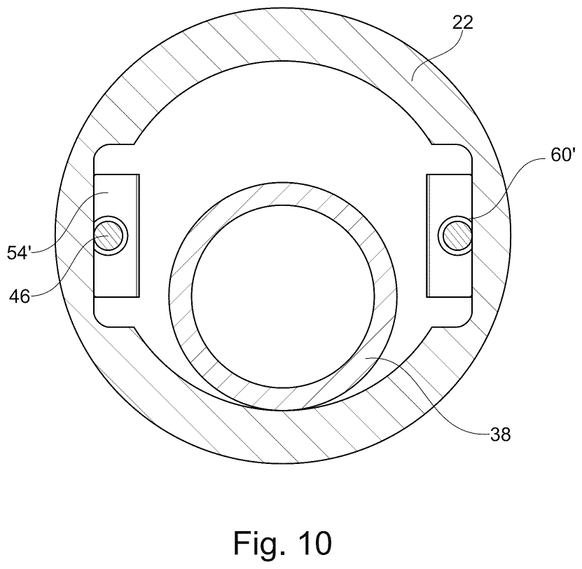

FIG. 10 shows a cross-sectional view of the bending section according to the variation of the alternative embodiment shown in FIGS. 9A and 9B but shown in an assembled compressed state of the bending section. The compressed bended webs 54′ are seen with the steering wires 46 passing through the holes formed by the apertures 60′. The working channel tube 38 is also shown in FIG. 10.

The bending section may be injection molded, may be manufactured with a 3D printing machine, or may be molded from polymers in any other way. In an injection molding process embodiment, the lumen 36 may be made with a mandrel. Side-action slides slide into position as the mold, which comprises two parts that together form the circumferential wall surfaces of the bending section, come together. When the mold is closed and the mandrels and side-action slides are in position, a melted polymer composition is injected into channels in the mold that are fluidly coupled with an injection molding extruder. The side-action slides form the spaces between the segments and the hinges. The mold may comprise a longitudinal protrusion to form the channel or may comprise a second mandrel to do so. Additional mandrels, or rods, can be used to form the steering wire passages. If the steering wire passages are open, a common mandrel can be used to form the steering wire passages and the central passage.

The following items are further variations and examples of the embodiments described with reference to the figures.
1. Endoscope (2) comprising: an endoscope handle or interface (4); and an insertion cord (6) configured to be inserted into a patient's body cavity and comprising an actively bendable bending section (10), the bending section (10) comprising an integral bending section body (16, 16′) made of polymer material, the bending section body (16, 16′) comprising a plurality of interconnected bending segments (18, 18′), wherein hinges (26, 26′) between adjacent bending segments (18, 18′) are formed by hinge protrusions (30, 30′) engaging in hinge recesses (32, 32′), and wherein flexible connection portions (34, 34′) are provided between the adjacent bending segments (18, 18′), for providing an interconnection between the adjacent bending segments (18, 18′).
2. Endoscope (2) according to item 1, wherein a hinge (26, 26′) between two adjacent bending segments (18, 18′) comprises two, preferably diametrically opposed, hinge interfaces (28, 28′), wherein each hinge interface (28, 28′) is formed by a hinge protrusion (30, 30′) engaging in a hinge recess (32, 32′).
3. Endoscope (2) according to item 2, wherein each hinge interface (28) is encircled or surrounded by two flexible connection portions (34), one flexible connection portion (34) being arranged clockwise in circumferential direction next to the hinge interface (28) and one flexible connection portion (34) being arranged anticlockwise in circumferential direction next to the hinge interface (28).
4. Endoscope (2) according to item 3, wherein the flexible connection portions (34) are spaced by less than 90° in circumferential direction, preferably by less than 45° in circumferential direction, from the hinge interface (28), in particular from a center of the hinge interface (28).
5. Endoscope (2) according to item 3 or 4, wherein the two flexible connection portions (34) encircling or surrounding the hinge interface (28) are symmetrically identical with respect to a bending plane defined by the two hinge interfaces (28) of the hinge (26).
6. Endoscope (2) according to any one of item 3 to 5, wherein the flexible connection portions (34) are curved or angled portions extending between two adjacent bending segments (18) and having a bend or inflexion point (50).
7. Endoscope (2) according to any one of items 3 to 6, wherein the flexible connection portions (34) are configured and provided to apply a force to the bending section body (16) which brings the bending section body (16) back to a neutral, unbent position.
8. Endoscope (2) according to any one of items 3 to 7, wherein the bending section body (16) comprises at least one inner lumen (36) and at least two steering wire lumen (44).
9. Endoscope (2) according to item 1 or 2, wherein two diametrically opposed flexible connection portions (34′) are provided between two adjacent bending segments (18′), each flexible connection portion (34′) being spaced by 90° in a circumferential direction from the hinge interfaces (28′).
10. Endoscope (2) according to item 9, wherein the flexible connection portions (34′) are designed and configured to provide guiding apertures (60′) for steering wires (46) in an assembled state of the endoscope (2), i.e. in a state in which the hinge protrusions (30′) engage the hinge recesses (32′).
11. Endoscope (2) according to item 9 or 10, wherein the guiding apertures (60′) have an oval shape or an elongated shape.
12. Endoscope (2) according to any one of items 1 to 11, wherein the hinge protrusions (30, 30′) engage the hinge recesses (32, 32′) only in an assembled state of the endoscope (2).
13. Endoscope (2) according to any one of item 1 to 12, wherein during assembly of the endoscope (2) the bending section body (16, 16′) is compressed via a pretensioning of steering wires (46), wherein the hinge protrusions (30, 30′) and the hinge recesses (32, 32′) are configured and provided to serve as compression limiting parts or portions, and wherein the flexible connection portions (34, 34′) are provided and configured to deform elastically.
14. Endoscope (2) according to any one of items 1 to 13, wherein the bending section body (16, 16′) is manufactured in an injection-molding process.
15. System comprising an endoscope (2) according to any one of items 1 to 14 and a monitor (13) connected to the endoscope (2).

The term “distal” is defined to be in the direction toward the patient, and the term “proximal” is defined to be in the direction away from the patient. For the handle of the endoscope, the distal end will be the end where the insertion tube is connected, and the proximal end is the opposite end.

A positioning interface functions to control the position of the insertion cord. A handle is an example of a positioning interface. The handle may include a steering wire actuator provided to operate the bending section. A robotic arm is another example of a positioning interface.

In the present disclosure, one steering wire is counted as a part extending from the steering wire actuator (or roller) to the distal end of the bending section. If an element extends from the steering wire actuator to the distal end of the bending section and back to the steering wire actuator it is counted as two steering wires and may be referred to as first and second steering wires. Thus, two steering wires may comprise distinct elements without an intermediate part connecting them at the distal end or parts of one element that includes an intermediate part connecting them at the distal end. A steering wire may comprise one string or a bundle of strings. The strings may be comprised or metal or polymer.

The use of the terms “first”, “second”, “third”, “fourth”, “primary”, “secondary”, “tertiary” etc. does not imply any particular order or importance. These labels are included to identify individual elements. Furthermore, the labelling of a first element does not imply the presence of a second element and vice versa.

As used herein, “in the range” or “between” or “in an interval” includes the values that define the range or interval. Therefore, “in the range of A-B” or “between A and B” or “in an interval of A to B” includes A and B.

As used herein, the terms “comprises,” “comprising,” or any other variation thereof, are intended to cover a non-exclusive inclusion, such that a process, method, article, or apparatus that comprises a list of elements does not necessarily include only those elements but may include other elements not expressly listed or inherent to such process, method, article, or apparatus. The term “exemplary” is used in place of “example” rather than “ideal.”

As used herein, the terms “about,” “substantially,” and “approximately,” indicate a range of values within +/−10% of a stated value.

The expression “actively bendable bending section” denotes that the bending section is structured to bend under control of a steering/bending mechanism. By contrast, a “passively bendable tube” may be flexible enough to bend, but the tube and the steering mechanism (including an operating unit and steering wires) are not configured to control such bending. It should be understood that the passively bendable tube may incur some bending as a result of the steering mechanism causing the actively bendable bending section to bend, where the bendable tube is located, and other factors such as lateral forces impinging on the passively bendable tube.

### LIST OF REFERENCE SIGNS

2 endoscope
4 endoscope handle
6 insertion cord
8 insertion tube
10 bending section
12 distal tip unit
13 monitor
14 cable
15 operating unit
16, 16′ bending section body
18, 18′ bending segment
20, 20′ proximal end segment
22, 22′ intermediate segment
24, 24′ distal end segment
26, 26′ hinge
28, 28′ hinge interface
30, 30′ hinge protrusion
32, 32′ hinge recess
34, 34′ flexible connection portion
36 inner lumen
38 working channel tube
40 electrical cable
42 tube
44 steering wire lumen
46 steering wire
48 upstream portion
50 bend/inflexion point
52 downstream portion
54′ longitudinal or axial web
56′ cross web
58′ predetermined bend or kink line
60′ aperture

1. An endoscope comprising:
a positioning interface; and
an insertion cord connected to and extending distally from the positioning interface, the insertion cord including an insertion tube and a bending section body that is actively bendable, the bending section body comprising a single piece of polymer material and extending from the insertion tube, the bending section body including:
flexible connection portions;
segments interconnected by the flexible connection portions; and
hinges between the segments, the hinges comprising hinge recesses and hinge protrusions configured to engage the hinge recesses.

2. The endoscope of claim 1, wherein the segments comprise pairs of the segments, the segments in each of the pairs being adjacent and interconnected by four flexible connection portions of the flexible connection portions, wherein each of the two hinge interfaces of the hinge is positioned between two of the four flexible connection portions, and wherein the hinge recesses are detached from the hinge protrusions.

3. The endoscope of claim 2, wherein each of the pairs of the segments comprises a distal segment and a proximal segment, wherein each of the flexible connection portions comprises a proximal portion connected to the proximal segment, a distal portion connected to the distal segment, and a curved portion connecting the proximal portion to the distal portion, the curved portion comprising an inflexion point.

4. The endoscope of claim 3, wherein the distal segment comprises the hinge recesses and the proximal segment comprises the hinge protrusions of the hinge.

5. The endoscope of claim 3, wherein the distal segment comprises the hinge recesses and the proximal segment comprises the hinge protrusions of the hinge, and wherein a plane passing through the inflexion points traverses the hinge protrusions of the hinge.

6. The endoscope of claim 5, wherein the curved portions connecting the proximal portions to the distal portions of the two flexible connection portions encircling the first hinge interface are spaced between 20° and 60°, in opposite circumferential directions, from a center of the first hinge interface.

7. The endoscope of claim 1, wherein a hinge of the hinges comprises two hinge interfaces, wherein each of the two hinge interfaces comprises a hinge protrusion of the hinge protrusions and a hinge recess from the hinge recesses, the hinge protrusion being disconnected from, and configured to engage with, the hinge recess.

8. The endoscope of claim 7, wherein the two hinge interfaces of the hinge are diametrically opposed.

9. The endoscope of claim 7, wherein the segments comprise a first segment and a second segment adjacent the first segment, the first segment and the second segment being interconnected by four flexible connection portions of the flexible connection portions, and wherein each of the two hinge interfaces is positioned between two of the four flexible connection portions.

10. The endoscope of claim 1, wherein the two hinge interfaces of the hinge comprise a first hinge interface and a second hinge interface, wherein the first hinge interface is encircled by two flexible connection portions of the flexible connection portions, one on each side of the first hinge interface, and wherein the second hinge interface is encircled by two flexible connection portions of the flexible connection portions, one on each side of the second hinge interface.

11. The endoscope of claim 10, wherein each of the two flexible connection portions encircling the first hinge interface is spaced by less than 90°, in opposite circumferential directions, from the first hinge interface, and wherein each of the two flexible connection portions encircling the second hinge interface is spaced by less than 90°, in opposite circumferential directions, from the second hinge interface.

12. The endoscope of claim 10, wherein each of the two flexible connection portions encircling the first hinge interface is spaced by less than 45°, in opposite circumferential directions, from a radial center the first hinge interface, and wherein each of the two flexible connection portions encircling the second hinge interface is spaced by less than 45°, in opposite circumferential directions, from a radial center the second hinge interface.

13. The endoscope of claim 10, wherein the two flexible connection portions encircling the first hinge interface are symmetrically identical with respect to a bending plane defined by the hinge.

14. The endoscope of claim 1, wherein the flexible connection portions are configured to apply a force to the bending section body which brings the bending section body back to a neutral, unbent position.

15. The endoscope of claim 1, wherein the bending section body comprises at least one inner lumen and two steering wire passages.

16. The endoscope of claim 15, wherein the two steering wire passages comprise closed peripheries.

17. The endoscope of claim 15, further comprising steering wires, wherein the flexible connection portions are configured to provide guiding apertures for the steering wires in a state of the endoscope in which the hinge protrusions engage the hinge recesses.

18. The endoscope of claim 17, wherein the guiding apertures have oval shapes or elongated shapes.

19. The endoscope of claim 1, wherein the hinge protrusions engage the hinge recesses only in an assembled state of the endoscope.

20. A system comprising:
the endoscope of claim 1; and
a monitor communicatively connected to the endoscope.
